(19) United States
(12) Patent Application Publication
(10) Pub. No.: US 2025/0152830 A1
(43) Pub. Date: May 15, 2025

(54) SMART INJECTION SYSTEM

(71) Applicant: Ypsomed AG, Burgdorf (CH)

(72) Inventors: Martin Brügger, Bolligen (CH); Patrick Hostettler, Hasle (CH); Stefan Mangold, Liebefeld (CH); Simon Schüpbach, Burgdorf (CH); Christian Schmid, Aarau (CH); Kuyoung Chung, Bern (CH); Benjamin Stettler, Bremgarten b. Bern (CH)

(21) Appl. No.: 19/028,153

(22) Filed: Jan. 17, 2025

Related U.S. Application Data
(63) Continuation of application No. PCT/EP2023/064649, filed on Jun. 1, 2023.

(30) Foreign Application Priority Data
Jul. 20, 2022 (CH) 000860/2022

(51) Int. Cl. A61M 5/31

(52) U.S. Cl. CPC A61M 5/31; A61M 2005/3126; A61M 2205/3379; A61M 2205/3592; A61M 2205/52; A61M 2205/584; A61M 2230/201

(57) ABSTRACT

A smart injection system includes at least one mobile device and at least one injection device with electronics that can be used by a user. The mobile device is configured to wirelessly receive information regarding a glycemic state of the user, use this information to automatically calculate medication doses to be administered, and to transmit the calculated doses to the injection device.

### CROSS-REFERENCE TO RELATED APPLICATIONS

This application is a continuation of International Patent Application No. PCT/EP2023/064649, filed Jun. 1, 2023, entitled “SMART INJECTION SYSTEM,” which in turn claims priority to Swiss Patent Application No. CH000860/2022, filed Jul. 20, 2022, entitled “SMART INJECTION SYSTEM”, each of which is incorporated by reference herein, in the entirety and for all purposes.

### TECHNICAL FIELD

The present invention relates to the field of liquid medication administration devices, in particular administration devices with electronics.

### BACKGROUND

Diabetes has become a widespread disease in recent years and the global prevalence is expected to rise to 783 million people worldwide by 2045. In comparison, the corresponding prevalence in 2002 was 151 million people. Type 1 diabetes cannot be cured to date, whereas type 2 diabetes can be partially reversed or at least slowed down in its early stages by changes in lifestyle. Many stages of diabetes are now-if not curable-at least well-treatable. The focus is on controlling blood glucose levels as well as possible in order to avoid long-term damage, such as vascular diseases.

In recent years, continuous or quasi-continuous systems for monitoring blood glucose levels, so-called CGM (Continuous Glucose Monitoring) systems, have become increasingly popular over conventional blood glucose measuring devices with test strips. CGM systems typically comprise a sensor unit that is attached to the skin. A sensor element protrudes subcutaneously from the sensor unit into the interstitial space of the person using the CGM system. The sensor unit (hereinafter also simply called CGM) detects the glucose concentration in the interstitium, which is then converted into an estimate of the blood glucose level. The measured values (or converted estimated blood glucose values) are transmitted wirelessly from the sensor unit to a receiver unit. The receiver unit is typically a handheld device, such as a smartphone, on which a suitable app is installed. The measured values can be evaluated in the app. In particular, the app can generate and issue messages, alarms, recommendations and/or instructions based on the measured values so that the user can keep their blood glucose level in the healthiest range possible. Instructions and recommendations may also comprise treatment instructions. For example, a recommendation for the administration of a correction dose of medication can be generated. The medication could be insulin or glucagon, for example. Alternatively, if necessary, a recommendation to intake carbohydrates can be given.

In modern diabetes management systems, the CGM system and medication delivery system can be linked in such a way that corresponding treatment instructions are automatically generated based on glucose data, automatically received by the medication delivery system and also automatically executed. A so-called closed loop is implemented. Such closed-loop systems with insulin pumps are established on the market; an example is the Minimed™ 780G system with Guardian™ 4 sensor (as CGM) from Medtronic. Such systems make everyday life much easier for patients with diabetes.

As a (simpler) alternative to insulin pump therapy, pen therapy remains an established way of treating diabetes with insulin. In typical settings, the patient uses two different types of insulin to easily cover different needs throughout the day. For example, one type may be a slow-acting insulin, so-called basal insulin. This insulin is administered once a day or less to meet basic insulin needs over a period of time. In order to cover the insulin requirement due to meals, snacks or unwanted blood glucose spikes, a fast-acting insulin (also called bolus insulin) is also used, which is administered as needed. In typical settings, the patient uses a first pen for the first type of insulin and a second pen for the second type. To avoid confusion, the two pens can differ in color, for example. Alternatively, different cartridge sizes can be used in the pens (1.6 mL or 3 mL). Since common injection pens, such as the Unopen from Ypsomed, the SoloStar® from Sanofi or the Flex-pen® from Novo Nordisk, are precision mechanical devices without electronic elements, it is very complicated to set up a closed-loop system using pens rather than insulin pumps. In the case of pens, the system is more of a decision support system.

From US20200350052 A1, a smart system is known which is composed of a CGM 50, a companion device 5 and an injection pen 10. The injection pen 10 comprises various electronic elements, wherein the pen 10 is operated manually for dose adjusting and dispensing, and the electronics can record the adjusted dose and transmit it wirelessly to the companion device 5, which continuously records the administrations. The companion device 5 also receives glucose data from the CGM. From the combination of administration data and glucose data, software on the companion device 5 can be used to create dosage recommendations. Because the companion device 5 continuously receives glucose data, it can also continuously adjust recommendations and trigger alarms when needed. However, in order to receive the dosage recommendation, the patient must pick up the companion device, read the recommendation (correctly) and finally adjust the injection pen accordingly. This potentially creates the risk that the patient may misread the dose. Another disadvantage is that the patient has to carry the companion device 5 in addition to the pen 10, which could potentially result in unwanted attention in a public setting. There is therefore a great need for further improvement of existing smart systems with pens.

### SUMMARY

It is an object of the invention to provide improved smart injection systems which allow for a safe, discreet and intelligent treatment of diabetes.

The object is achieved by the systems and the methods of the present disclosure.

One aspect relates to a system comprising a liquid medication administration device and a mobile computer also referred to as a mobile device. The administration device can advantageously be an injection device, especially a pen-shaped injection device, such as an injection pen or an auto-injector. It should be noted that administration apparatuses are known from the prior art which consist of, for example, an injection pen or an auto-injector and, in addition, an add-on module. Such device combinations are explicitly not intended as the administration device according to the invention. In preferred variants, the liquid medications are medications for the treatment of diabetes, in particular forms of insulin or GLP-1 analogue medications. The mobile device can advantageously be a handheld computer, a mobile phone, a smartphone, a smart watch, a notebook, a tablet or any other suitable portable electronic device. The mobile device comprises the typical elements of a computer or smartphone as they are well known to a person skilled in the art on the filing date of this application. In particular, the computer comprises a processor which controls the computer. For this purpose, software (including an operating system or firmware) is installed on the mobile device, which can be executed on the hardware (especially the processor). The mobile device also comprises modern wireless communication options, such as Bluetooth, WLAN, mobile radio (3G, 4G or 5G) and/or NFC, via which the mobile device can receive and transmit information and/or data. In particular, the mobile device can receive glucose data via wireless communication capabilities. These data can come directly from a blood glucose measuring device or a (quasi-)continuously measuring glucose measuring device, also known as a CGM device. Alternatively, the data may originate from another device, in particular a server, a remote control for CGMs, or another computer that receives data directly or indirectly from the CGM. Furthermore, mobile devices as they are known typically comprise various signal transmitters, for example displays, light-emitting diodes (LEDs), tactile signal transmitters and/or acoustic signal transmitters such as loudspeakers or buzzers.

The administration device comprises in particular a dose adjusting element with which the dose can be manually adjusted by a user, wherein the adjusted dose is visible on a display. The administration device also comprises electronics, in particular a processor or controller configured as a central control unit. A communications module for wireless communication is communicatively coupled to the processor or controller. The module is advantageously a Bluetooth, NFC, WLAN module or a combination of these. The module is designed to transmit or receive information or data, in particular from the mobile device. The administration device also comprises a signal module. The signal module is also connected to the processor or controller and controlled by it. The signal module serves to signal states of the administration apparatus and/or events in or on the administration device to the user. The signaling apparatus may consist of one or more LEDs, in particular a red-green-blue (RGB) LED. Alternatively or in addition, tactile and/or acoustic signal transmitters can also be used for the signaling apparatus. LCD, E-Ink, OLED or similar displays are also possible.

As mentioned, the mobile device can receive glucose data wirelessly. These data typically come from the user and provide information about the user's current glycemic state. The glucose data can be processed via the software installed on the mobile device and running on the processor. This is in particular useful for calculating medication doses. For example, the most recent glucose measurement (blood glucose or interstitial glucose value) can be used to calculate a correction dose with a fast-acting insulin—a so-called correction bolus. It seems important to mention again at this point that the correction dose is recalculated based on received data and is not predefined in its volume or derived from a dose schedule. According to the invention, the mobile device and the installed software are designed to automatically calculate whether a correction dose is necessary or not when current glucose values are received—and if so, also to calculate how large the correction dose of the medication should be. Preferably, if a calculated dose is below a predetermined threshold, the value for the calculated dose is simply stored (or discarded) and the system waits for the next incoming glucose values. If the calculated dose exceeds the threshold, the volume of the dose is automatically transmitted to the administration device via wireless communication options, for example via Bluetooth. Alternatively, it is also possible that there is no predetermined threshold, and small doses are also transmitted to the administration device. At the same time, one of the signal transmitters of the mobile device can preferably emit a signal to the user, indicating that a correction bolus—or, more generally, a correction dose—should be administered, wherein the signal is preferably only emitted when the dose has reached a certain level. This signal can, for example, be a discreet vibration of the mobile device. According to the invention, the user can then hold the administration device, on which the calculated dose has already been received, and adjust the dose via the dose adjusting element, wherein the processor or controller monitors the adjusting process and continuously compares the adjusted dose with the calculated dose. If the adjusted dose reaches the volume of the calculated value, the processor or controller causes the signaling apparatus to emit a signal which signals to the user that the dose calculated by the mobile device has now been reached and set. If the calculated dose is exceeded, the processor or controller can preferably issue another, different signal to signal the user that he or she should correct the dose downwards. Once the calculated dose has been adjusted, nothing stands in the way of administration. If the person using the device does not agree with the volume of the dose, he or she can administer a dose other than the one set. Advantageously, the processor or controller stores the calculated dose and the actually-administered dose together. In the administration device described in this example, the dose is manually adjusted. However, it is also conceivable that in an alternative, the dose is already adjusted when the dose is transmitted to the administration device, and the person using it can, if he or she agrees with the predetermined dose, proceed directly to administration.

In an advantageous embodiment, after administration, the calculated value and the actually-administered value are transmitted to the mobile device via the wireless communication module. This can happen immediately after administration or at defined times. Alternatively, the transmission can also be done easily when a wireless connection is established with the mobile device. The processor or controller is advantageously equipped with a sufficiently large memory for values so that, for example, all values for one week can be stored on the administration device (for example 300 values).

The system described has multiple advantages. The automatic calculation of correction doses and the subsequent automatic transmission of the doses to the administration device allow the user to administer a correction dose very discreetly. This means that the user does not have to open an app on their mobile device and manually initiate the calculation of the correction dose. Also, the patient does not have to manually transmit the calculated dose to the administration device, but can simply take the administration device, adjust the dose until the corresponding signal is given by the administration device, and then administer the dose. The signal when the calculated dose is reached can, for example, be a green LED lighting up or emitting a green light. In addition to the useful discretion, the described system also has a safety advantage over known systems. Since the calculated dose is transmitted automatically, the probability of incorrect manipulation by the user can be reduced. Setting the correct dose is also made easier for visually impaired people, as a visual, audible or tangible signal is given when the calculated dose is reached. Advantageously, the signal is designed so simply that the user does not have to read the adjusted dose from the administration device, or the signal comprises a display of text.

In one aspect of the invention, the system described above further comprises a (quasi-)continuously measuring tissue glucose level sensor (hereinafter referred to as CGM), which, like the administration device, comprises a module for wireless communication. The CGM device is applied directly to the skin of the user and comprises a sensor that is inserted subcutaneously into the tissue. Such sensors are known to professionals from various manufacturers, such as Dexcom or Abbott Diabetes Care. For example, the CGM device can transmit a glucose measured value every five minutes to the mobile device, which then processes the measured value as described above. Further processing can be done based on individual values. Alternatively, multiple measured values can be aggregated before further processing, for example by averaging or filtering (in particular via a Kalman filter), in order not to overestimate short-term fluctuations in the measurement series.

In one aspect of the invention, the system comprises not just one administration device, but multiple. All of the system's native administration devices can communicate wirelessly with the mobile device as discussed above for a system with one administration device. Preferably, the administration devices differ primarily in the medications that are administered with the individual administration devices; otherwise, the devices can be technically identical and function in the same way as described above. The following describes an example of a system according to the invention which comprises two administration devices. In this example, the mobile device is a smartphone that can communicate with the two administration devices via Bluetooth. The two administration devices according to the invention are, in the example, injection pens into which a medication reservoir is inserted, in particular a carpule. Technically, both pens are about the same and meet the description above. In the first administration device, the reservoir contains rapid-acting insulin or bolus insulin, such as insulin lispro. In the second administration device, the reservoir contains slow-acting insulin or basal insulin, such as insulin glargine. When treating diabetes with insulin, one of the common variants is to combine slow basal insulin with bolus insulin for meals and correction boluses. For example, a diabetes patient can take a basal dose in the morning or evening, and can administer bolus insulin during meals and blood glucose spikes throughout the day, which acts faster than the basal insulin. Such forms of therapy are known to a person skilled in the art. The software on the smartphone can calculate and record doses for both pens.

In a further aspect of the invention, which further develops the previous aspect, the administration devices comprise real-time clocks which are connected to the processor or controller of the respective administration devices. This means that in addition to the calculated dose and the actually-administered dose, the exact time (with date) can also be saved and/or transmitted to the mobile device. This additional functionality is very interesting when accidental repeated administration of a dose is to be prevented. As an example, the injection pen with basal insulin is used again. Basal insulin is typically administered in one or two doses per day. However, it can happen that the person using it has forgotten that he or she has already administered the morning basal insulin and wants to administer the dose a second time. This can be prevented in embodiments according to the invention by starting a timer after administration of basal insulin by the processor or controller and taking into account the real-time clock, for example for eight or 20 hours in the case of basal insulin. If the user tries to set a basal dose while the timer is still running, the processor or controller detects this and can issue a corresponding signal via the signal module. If the signal module comprises an LED arrangement, a red flashing light can, for example, signal that there is a risk of double dosing. The user can then check on the mobile device whether and which dose has already been administered. This can also work analogously for fast-acting insulin, although the timer would of course be activated for shorter periods of time. In one embodiment of the invention, the mobile device can be configured in such a way that it can deactivate or override running timers on the administration devices so that, for example, in the case of extremely high measured glucose levels, a further dose can be administered before the timer expires without a corresponding “error signal.”

In a further aspect of the invention, the software comprises a so-called bolus calculator for meals, to which a meal database is attached. In one example, the database can be simple and contain in particular fat, protein and carbohydrate contents of important staple foods. Alternatively, a detailed meal database can be stored, as is known from codecheck, for example. Before a meal, the user uses the database to indicate what he or she intends to eat during the meal. Based on the amount and type of food entered, the bolus calculator analyses the ratio of protein, fat and carbohydrates in the meal. If the fat content exceeds a certain level, it is to be expected that the meal will be digested slowly. In this case, the bolus calculator divides the total insulin dose into partial doses, which should be administered over a certain period of time. The mobile device's processor then transmits a single partial dose to the administration device at the appropriate time.

### BRIEF DESCRIPTION OF THE DRAWINGS

Preferred embodiments of the invention are described below in connection with the appended figures. These embodiments are intended to show basic possibilities of the invention and are in no way to be interpreted as limiting.

FIG. 1 shows a schematic of possible user who uses the system according to the present disclosure, including a CGM, a smart pen and a smartphone;

FIG. 2 shows the smart pen of FIG. 1; and

FIG. 3 shows the system including the smartphone, the smart pen, a second smart pen, and the CGM.

### DETAILED DESCRIPTION

### Definitions

The term “product,” “medication,” or “medical substance” in the present context comprises any flowable medical formulation which is suitable for controlled administration by means of a cannula or hollow needle in subcutaneous or intramuscular tissue, for example a liquid, a solution, a gel, or a fine suspension containing one or more medical active ingredients. A medication can thus be a composition with a single active ingredient or a premixed or co-formulated composition with a plurality of active ingredients from a single container. The term includes in particular drugs, such as peptides (e.g., insulins, insulin-containing medications, GLP 1-containing preparations as well as derived or analogous preparations), proteins and hormones, biologically obtained or active ingredients, active ingredients based on hormones or genes, nutrient formulations, enzymes, and other substances both in solid (suspended) or liquid form. The term also includes polysaccharides, vaccines, DNA or RNA or oligonucleotides, antibodies or parts of antibodies as well as suitable base substances, excipients, and carrier substances.

The term “distal” refers to a side or direction directed toward the front, piercing-side end of the administration apparatus or toward the tip of the injection needle. In contrast, the term “proximal” refers to a side or direction directed toward the rear end of the administration apparatus that is opposite the piercing-side end.

In the present description, the terms “injection system”, “injection device”, “injection pen” or “injector” are understood to mean an apparatus in which the injection needle is removed from the tissue after a controlled amount of the medical substance has been dispensed. In contrast to an infusion system, the injection needle in an injection system or in an injector thus does not remain in the tissue for a longer period of several hours.

The term “environment” is used in the context of this document when an item, such as an administration device, emits a signal outside the item that is visible, audible, palpable or measurable from outside the item. Thus, the term “environment” primarily refers to the immediate surroundings of the item.

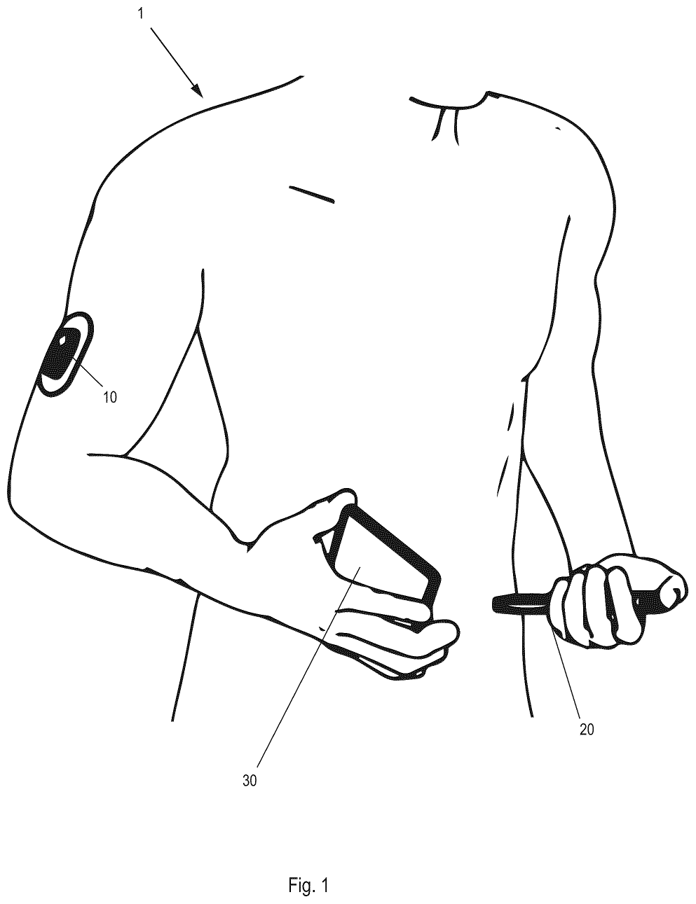

FIG. 1 shows a system according to the invention as it could be used. The user 1 is currently administering a dose of medication (not shown) with the smart pen 20. The administered dose was previously calculated using the smartphone 30 based on glucose data received from the CGM 10 and automatically passed on to the smart pen.

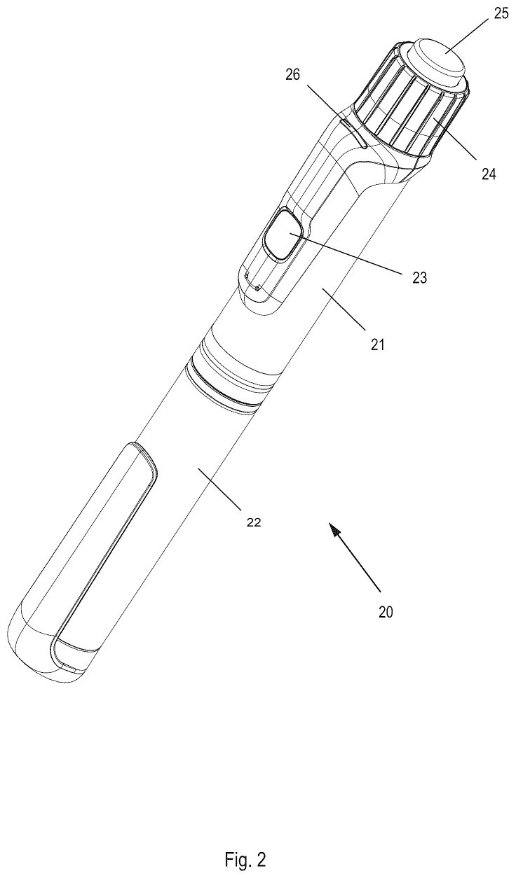

FIG. 2 shows the smart pen 20 in an external view. The smart pen 20 comprises in particular the housing 21, the pen cap 22, the display 23, the dosing element 24 and the injection button 25. Furthermore, the smart pen 20 also comprises an LED 26, which may in particular comprise an RGB LED (not shown). The display 23 is advantageously an electronic display in the form of an LCD or OLED display. Various of the electrical and electronic elements described above are not shown in this view-but the smart pen 20 also comprises all the described elements such as a processor or controller, wireless communication module, signal module, etc. If a calculated dose (as shown in the example in FIG. 1) is received in the smart pen, this can be indicated via the LED 26, for example by the LED 26 flashing blue. Alternatively, the smart pen 20 can also vibrate temporarily in one embodiment. In another alternative, signaling can be dispensed with. In order to administer a received dose from the smart pen 20, the user 1 grasps the smart pen 20 and rotates the dosing element 24 in a first direction, wherein the adjusted dose is continuously displayed on the display 23. If the level of the received dose is adjusted, the LED 26 lights up green in an advantageous example. If the user 1 turns the dosing element 24 further in the first direction, the LED 26 begins to light up or flash orange in an advantageous example. If the user 1 then rotates the dose adjusting element 24 in a second direction, which is opposite to the first direction, to correct the adjusted dose, the LED 26 in the example starts to light up green again as soon as the received dose is reached again. As soon as the user 1 has set the dose that he or she actually wants to administer (this may well deviate from the transmitted calculated dose), he or she removes the pen cap 22, places an injection cannula (not shown) on the distal end of the smart pen 20, inserts the cannula into the tissue and triggers the injection (i.e., the administration of the set dose) by pressing the injection button 25 in the distal direction. In one example, after the entire dose has been administered, the LED 26 may begin to flash green to indicate to the user that the cannula can be removed from the tissue. The actually-administered dose is stored in the smart pen 20 and can be transmitted wirelessly to the smartphone 30 immediately after administration or at a later time. The smartphone stores the data on the transmitted administered doses together with the corresponding calculated doses. This stored data can then be included in the calculation of future doses.

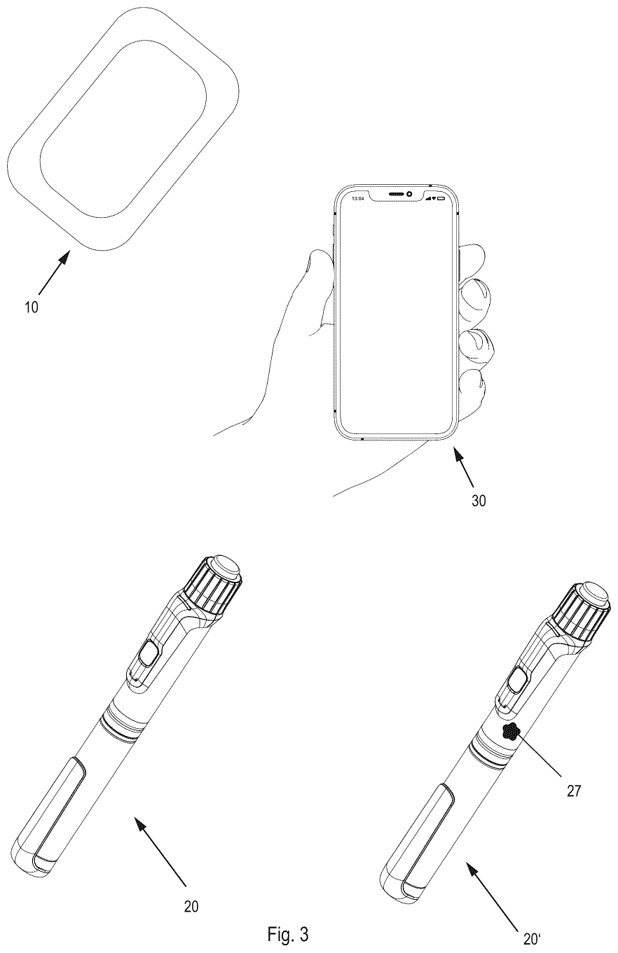

FIG. 3 shows the elements of another system in which two smart pens 20, 20′ are used. The smart pen 20 and the second smart pen 20′ differ only in the medications used in the pens and the marking(s), represented by the symbolic distinction 27 between the smart injection pens 20, 20′. Otherwise, both injection pens 20, 20′ have identical features, which means that the description of the elements of the smart pen 20 in the description of FIG. 2 also applies to the smart pen 20′. For reasons of clarity, separate designation of the elements has been omitted. The system in FIG. 3 comprises the injection pens 20, 20′, the smartphone 30 and the CGM 10. In the system shown in FIG. 3, the medication that can be administered with the smart pen 20 is a (very) fast-acting insulin that can be used during meals or glucose spikes. On the other hand, the second smart pen 20′ administers a slow-acting insulin, a so-called basal insulin. Both injection pens 20, 20′ can communicate wirelessly with the smartphone. The smartphone 30 can distinguish the two injection pens 20, 20′ from each other via specific identifiers and address them separately.

An app (not shown) is installed on the smartphone 30, with which the entire system can be coordinated and controlled. The app processes the data received from, for example, the CGM 10 and uses this data to calculate the doses of insulin to be administered. In the system as shown in FIG. 3, the app distinguishes whether a basal dose or a bolus dose is calculated.

Typically, a basal dose is administered once a day, in the morning. For this purpose, the app calculates the basal dose to be administered at a specific time of day and then automatically transmits it to the second smart pen 20′. The procedure or the administration process is then also carried out with the second smart pen 20′ as shown in the description, for example in FIG. 2. Since the second smart pen 20′ is typically used only once or perhaps twice per day, in one example a real-time clock (not shown) is present on the second smart pen 20′. This clock can be used to prevent the user 1 from administering too many doses of basal insulin. For example, if a full day dose has been received from the smartphone 30 and subsequently administered, if the user sets another dose within a predetermined period of time (e.g., 12 or 22 h), the LED 26 of the second smart pen 20′ may begin to glow red. This is a simple way to signal to the user that a further basal dose is not yet indicated from the system's point of view. However, the user is free to administer another dose anyway; the adjusted dose is displayed at any time on the display 23 of the second smart pen 20′. An additional basal dose that is actually administered is then also stored on the second smart pen 20′ and transmitted to the smartphone 30.

The smart pen 20 is used in the system shown in FIG. 3 to deliver insulin boluses. The app on the smartphone 30 can continuously receive (glucose) data from the CGM 10 and also receives the administration data from the injection pens 20, 20′, which means that the app can also continuously determine the current need for insulin. Advantageously, the app also comprises a so-called bolus calculator. This bolus calculator can be used to calculate a meal bolus and/or correction bolus. Known bolus calculator routines include parameters such as current glucose concentration, insulin on board and any planned amount of carbohydrates entered by the user. The bolus calculator then calculates the bolus dose to be administered. In an advantageous embodiment, the user not only enters the planned amount of carbohydrates, but also sets how heavy the planned meal will be, for example by additionally entering a rough fat content. If the heaviness of the food exceeds a certain threshold, the app does not calculate a single dose, but rather two or more partial doses to take into account the fact that heavy foods, such as pizza or cheese fondue, are digested more slowly than lighter foods, such as white bread. The partial doses are transmitted from the smartphone 30 to the smart pen 20 at staggered times. For example, a first partial dose can be transmitted to the smart pen 20 immediately after it has been calculated (from where it can also be administered straight away). For example, a second partial dose can be transmitted one hour after the calculation and a possible third dose two hours after its calculation. Alternatively, all three partial doses can be transmitted to the smart pen 20 directly after they have been calculated, and a clock in the smart pen 20 can then initiate a signaling to the person using it according to the app's specifications at the right time. The actual administration procedures have already been explained in the description of FIG. 2.

In an advantageous embodiment of the injection pens 20, 20′ according to the invention, the electrical and/or electronic components of the injection pens 20, 20′ can be supplied with energy from an accumulator (rechargeable battery) (not shown). The battery can be charged inductively (i.e., wirelessly) or wired via a charging port, in particular a USB-C port. In addition, the system of this embodiment additionally comprises a docking station (not shown) into which the injection pens 20, 20′ can be plugged in and charged-either only one pen can be plugged in at a time or there can be multiple docking ports for multiple pens. In an advantageous variant, the docking station can also be used to load data from the injection pens 20, 20′ to the docking station or to load data from the docking station to the pens 20, 20′. In the former case, for example, the administration history can be loaded from a pen onto the docking station. In the latter case, for example, a firmware update can be loaded from the docking station onto a pen and installed there. The docking station is connected directly or via a network to the smartphone 30 and/or other computers/servers in order to receive and transmit data. The data connection between the docking station and the device can be wired, in particular via a USB-C port if available, or wirelessly using technologies known to a person skilled in the art.

In an advantageous embodiment, the smartphone 30 comprises a sensor for detecting movements, for example an acceleration sensor. The sensor signal can be included in the app's data processing. This allows the app, for example, to recognize athletic activities and (automatically) adjust the target glucose value accordingly. The app could also detect when a user sits down based on a movement pattern. Together with the current time, the app can deduce that the user has perhaps sat down to eat and then (automatically) suggest a pre-meal bolus and/or automatically transmit it to the smart pen 20.

### LIST OF REFERENCE SIGNS

1 user
10 (quasi-)continuous tissue glucose level sensor/CGM
20 smart injection pen/smart pen
20° second smart injection pen/second smart pen
21 housing
22 pen cap
23 display
24 dosing element
25 injection button
26 LED
27 symbolic distinction between the smart injection pens
30 smartphone/mobile device

What is claimed is:

1. A system, comprising:
a mobile device comprising a processor and a wireless communications module communicatively coupled to the processor, wherein the wireless communications module is configured to wirelessly receive blood glucose values from blood glucose measuring devices or glucose values from continuously or quasi-continuously measuring devices that measure interstitially in tissue; and
an administration device configured for administering a liquid medication in which a volume of a dose to be administered can be manually adjusted, wherein the administration device comprises a processor and a wireless communications module and a signal module communicatively coupled to the processor, the processor configured to cause the signal module to emit signals to an environment,
wherein the processor of the mobile device is configured to calculate a dose to be administered based at least on received blood glucose values or received glucose values, and cause the calculated dose to be wirelessly transmitted to the administration device via the communications module of the mobile device, and
wherein the processor of the administration device is configured to compare a manually adjusted dose to the calculated dose received from the mobile device, and, if the volume of the manually adjusted dose and a volume of the calculated dose are the same, the processor causes the signal module to emit a first signal to the environment.

2. The system according to claim 1, further comprising a continuously or quasi-continuously measuring tissue glucose level sensor configured to wirelessly transmit at least the measured glucose values to the mobile device.

3. The system according to claim 1, wherein the administration device is a first administration device, the system further comprising a second administration device configured for administering a liquid medication in which a volume of a dose to be administered can be manually adjusted, wherein the second administration device comprises a processor and a wireless communications module and a signal module communicatively coupled to the processor, the processor configured to cause the signal module to emit signals to the environment,
wherein the first administration device is configured to administer bolus insulin, and the second administration device is configured to administer basal insulin,
wherein the mobile device is configured to calculate doses of bolus insulin for delivery by the first administration device and doses of basal insulin for delivery by the second administration device and to transmit the calculated doses to respective administration devices, and
wherein the processor of the second administration device is configured to compare a manually adjusted dose of the second administration device with a calculated dose of basal insulin received from the mobile device and, if the volume of the manually adjusted dose and a volume of the calculated dose are the same, the processor causes the signal module to emit a second signal to the environment.

4. The system according to claim 3, wherein the first administration device and the second administration device are both configured to wirelessly transmit dose volumes of actually-administered doses of the liquid medications to the mobile device.

5. The system according to claim 4, wherein at least the second administration device further comprises an electronic clock module and memory configured for storing a time, a type and a volume of actually-administered doses of the liquid medication, and wherein the processor of the second administration device is configured to detect an administration of medication via communicatively coupled sensors, wherein the processor of the second administration device is configured to cause the detected administration to be stored with a current time, and simultaneously causes a timer to be started for a limited period of time, and wherein the processor of the second administration device is configured cause the signal module to emit a third signal to the environment when a further dose is set during the limited period of time.

6. The system according to claim 5, wherein the signal module of the second administration device comprises a tactile signal transmitter, an acoustic signal transmitter or an optical signal transmitter.

7. The system according to claim 6, wherein the signal transmitter is configured as the optical signal transmitter with a light-emitting diode arrangement that can emit light in different colors.

8. The system according to claim 7, wherein an emission of a green light by the light-emitting diode arrangement corresponds to the second signal and an emission of a red light corresponds to the third signal.

9. The system according to claim 1, wherein the signal module of the administration device comprises a tactile signal transmitter, an acoustic signal transmitter or an optical signal transmitter.

10. The system according to claim 9, wherein the signal transmitter is configured as the optical signal transmitter with a light-emitting diode arrangement that can emit light in different colors.

11. The system according to claim 10, wherein an emission of a first color by the light-emitting diode arrangement corresponds to the first signal.

12. The system according to claim 1, wherein the processor of the mobile device is configured to receive historical administration data from the administration device.

13. The system according to claim 1, wherein at least one of the administration device or the second administration device is configured as an injection device.

14. A method for providing an automatically calculated liquid dose of medication, comprising:
receiving blood glucose or glucose measured values on a mobile device of a system,
using a processor of the mobile device for calculating a dose to be administered at least based on stored blood glucose or glucose values and on received blood glucose or glucose values, and
wirelessly transmitting to an administration device of the system, the calculated dose to be administered.

15. The method according to the claim 14, wherein the administration device is a first administration device, wherein the system further comprises a second administration device, wherein the calculated dose is for either the first administration device or for the second administration device, and wherein the calculated dose is transmitted to a respective administration device for which the dose was calculated.

16. The method according to claim 14, wherein calculating the dose to be administered is based on historical administration data stored on the mobile device, the historical administration data received from the administration device prior to calculating the dose to be administered.
